SEQUENCE LISTING (23 sequences)

SEQ ID NO: 1 - DNA, 97 bp, Artificial
  Chemically synthesized
ttgtgcaaatcctgagactcatcaaacccatgaaagacggtacaaggtatactggaatccgatctctgaaacttgacatgaacccaggcactggtat

SEQ ID NO: 2 - DNA, 108 bp, pig
gggtggtggagaggggtgaattcgtccctctctgcctagtgggaggaaaatgaggggttccggagtattgaggccaaccgaagatgctatcaggtgagtgtaaactga

SEQ ID NO: 3 - DNA, 193 bp, cattle
acattctctacaccaagagaatcaagcacgaaagttattatgaaaccaataaccaaaggaggatttagcagtaaactaagaatagagtgcttagttgaattaggccatgaagcacgcacacaccgcccgtcaccctcctcaaatagattcagtgcatctaaccctatttaaacgcactagctacatgagagga

SEQ ID NO: 4 - DNA, 123 bp, sheep
cctctccagtgctgacttggatcttggggtacttctggagtctccccaggggagtcagttctcctctcgagagggggcatgcacgtgcgctttcctcccgaacttagcagcaatgtcatgctt

SEQ ID NO: 5 - DNA, 190 bp, deer
catttattatcgcagcactcgctatagtacacttactcttccttcacgaaacaggatctaataacccgacaggaattccatcagacgcagacaaaatcccctttcatccttattataccattaaagatatcttaggcatcttacttcttgtactcttcttaatatcactagtattattcgtaccagacct

SEQ ID NO: 6 - DNA, 99 bp, horse
gatggagagaaatgggctacattttctaccctaagaacaagaactttaacccggacgaaagtctccatgaaactggagactaaaggaggatttagcagt

SEQ ID NO: 7 - DNA, 106 bp, kangaroo
gagcttaattgaaacaggcaatagggcgcgcacacaccgcccgtcaccctcctcgacaaaaccttacaaataactaatacaacggaatacaaaagaggagaaaagt

SEQ ID NO: 8 - DNA, 31 bp, pig
cctcaatactccagaacccctcattttcctc

SEQ ID NO: 9 - DNA, 26 bp, cattle
acattctctacccaagagaatcaagc

SEQ ID NO: 10 - DNA, 25 bp, cattle
tcctctcatgtagctagtgcgttta

SEQ ID NO: 11 - DNA, 33 bp, cattle
ccctcctcaaatagattcagtgcatctaaccct

SEQ ID NO: 12 - DNA, 21 bp, sheep
cctctccagtgctgacttgga

SEQ ID NO: 13 - DNA, 24 bp, sheep
aagcatgacattgctgctaagttc

SEQ ID NO: 14 - DNA, 21 bp, sheep
cacgtgcatgccccctctcga

SEQ ID NO: 15 - DNA, 23 bp, deer
catttattatcgcagcactcgct

SEQ ID NO: 16 - DNA, 27 bp, deer
aggtctggtacgaataatactagtgat

SEQ ID NO: 17 - DNA, 28 bp, deer
ccacttactcttcctccacgaaacagga

SEQ ID NO: 18 - DNA, 25 bp, horse
gatggagagaaatgggctacatttt

SEQ ID NO: 19 - DNA, 27 bp, horse
actgctaaatcctcctttagtctccag

SEQ ID NO: 20 - DNA, 31 bp, horse
accctaagaacaagaactttaacccggacga

SEQ ID NO: 21 - DNA, 20 bp, kangaroo
gagcttaattgaaacaggca

SEQ ID NO: 22 - DNA, 23 bp, kangaroo
acttttctcctcttttgtattcc

SEQ ID NO: 23 - DNA, 18 bp, kangaroo
tcctcgacaaaaccttac

(19) United States
(12) Patent Application Publication
(10) Pub. No.: US 2007/0009910 A1
(43) Pub. Date: Jan. 11, 2007

(54) Primers, probes and reference plasmid for detection of meat adulteration

(75) Inventors: Lih-Ching Chiueh, Taipei City (TW); Yu-Chih Chen, Taipei City (TW); Shiou-Wei Tsuei, Taiping City (TW); Tsung-Hsi Wu, Taipei City (TW); Shin-Shing Li, Tainan County (TW); Chun-Hsien Li, Sindian City (TW); Yuan-Hsin Chang, Taipei City (TW); Yang-Chih Shih, Taipei City (TW); Shu-Kong Chen, Sindian City (TW)

Correspondence Address: HUSCH & EPPENBERGER, LLC, 190 CARONDELET PLAZA, SUITE 600, ST. LOUIS, MO, 63105-3441, US

(73) Assignee: Bureau of Food and Drug Analysis, Department of Health, Executive Yuan, Taipei City (TW)

(21) Appl. No.: 11/174,786

(22) Filed: Jul. 5, 2005

(30) Foreign Application Priority Data
Jul. 5, 2004 (TW) 093120150

(51) Int. Cl. C12Q 1/68; C07H 21/04; C12N 1/00

(52) U.S. Cl. 435/6; 435/317.1; 536/23.2

(57) ABSTRACT

The present invention relates to a reference plasmid for use in specifically detecting the meat of pig, cattle, sheep, deer, horse and kangaroo. The reference plasmid can be used in rapidly identifying the species of meat and can be used in testing commercial meat products.

### FIELD OF THE INVENTION

The present invention relates to a reference plasmid for use in specifically detecting pig, cattle, sheep, deer, horse and kangaroo meat. The reference plasmid can be used in rapidly identifying the species of meat and can be used in testing commercial meat products.

### BACKGROUND OF THE INVENTION

The identification of meat adulteration is a significant task. Unfaithful businessmen, in order to obtain colossal, illegal profits, mix cheap food into expensive food for sale. For example, it was found in Taiwan that the meat of kangaroo and marine turtles was mixed into commercial frozen beef. It was also found in Taiwan that there was pork in commercial beef jerky, or beef jerky was displaced by horse or ostrich meat. In addition, in Japan, Matusaka bull meat, which is cheaper, was mixed into Matusaka cow meat, which is more expensive. Thus, it is necessary to test meat products to identify the quality of meat therein and ensure the value thereof.

Generally speaking, the conventional methods for identifying the species of meat mainly utilize morphology, protein methods (e.g. one-dimensional protein electrophoresis technique and immunoserological antigen-antibody assay) and chemical methods (e.g., High Performance Liquid Chromatography), etc. However, protein denaturation often occurs in animal meat during the manufacturing process such that the above various morphological identifications, protein methods and chemical methods are not able to identify the species of meat effectively. Recently, with the development of molecular biological techniques, the above problems can be solved by efficiently utilizing DNA-based detection techniques to detect a small amount of sample DNA. The methods based on molecular biology comprise, for example, DNA hybridization (Trends in Food Science & Technology. 11:67-77) and PCR product sequencing for identification of e.g., tuna (J. Agric. Chem. 50:963-969); PCR-restriction fragment length polymorphism (PCR-RFLP) for identifications of e.g., pig, cattle and sheep (J. Agric. Food Chem. 51:1771-1776; J. AOAC Int. 78:1542-1551; J. Agric. Food Chem. 51:1524-1529; and J. Food Prot. 66:682-685), and identifications for puffer fish and frozen fish steak; species-specific primers PCR for identifications for e.g., pig, cattle, sheep, chicken and horse (J. Food Prot. 66:103-109; J. Agric. Food Chem. 49:2717-2721; and Meat Sci. 51:143-148); PCR-SSCP for identification of e.g., fish (Food Chem. 64:263-268); random amplified polymorphic DNA (RAPDs) for identification of e.g., clams and poultry (J. Agric. Food Chem. 50:1780-1784; and Poult Sci. 80:522-524); actin for identification of e.g., chicken (Meat Sci. 53:227-231); real-time PCR, such as real-time PCR employing the TaqMan Probe System to detect beef products (Bundesgesundheitsblatt Gesundheitsforsch. Gesundheitsschutz. pp.1-25); DNA-Chips, and the like. However, these methods still have limitations in identifying the species of meat. Take the most popular PCR, PCR product sequencing and PCR-RFLP methods for instance. The PCR method only make the identification based on the size of the PCR-amplified product, without any confirming step, which is the defect in the method, while the PCR product sequencing method must rely on the sequencing comparison, thus requiring equipment and techniques that a general detection laboratory cannot afford, and the PCR-RFLP method must find the proper restriction enzyme.

In addition, a challenge for meat identification methods is the difficulty to detect various species of meat. Although standards for some species are commercially available, they are only used for identification of DNA of a single species; for example, for identification of red deer DNA. Furthermore, the selection of target genes is another challenge. Actually, to determine whether target genes arise from the same or different species is the bottleneck in the development of identification methods.

Therefore, it is still necessary to develop a simple, rapid and practical method for identifying the species of meat.

### SUMMARY OF THE INVENTION

The present invention provides a reference plasmid for identifying the species of meat, comprising a meat internal control gene and the following nucleic acid sequences: the porcine growth hormone gene, the bovine 12S ribosomal RNA gene, the ovine satellite DNA, the cervine mitochondrial cytochrome b gene, the equine 12S ribosomal RNA gene and the kangaroo 12S ribosomal RNA gene.

The present invention also provides a method and a kit for identifying the species of meat.

### BRIEF DESCRIPTION OF THE DRAWING

FIG. 1 shows the first PCR-amplified DNA fragment during the construction of the reference plasmid for detection of meat according to the present invention.

FIG. 2 shows the second PCR-amplified DNA fragment during the construction of the reference plasmid for detection of meat according to the present invention.

FIG. 3 shows the third PCR-amplified DNA fragment during the construction of the reference plasmid for detection of meat according to the present invention.

FIG. 4 shows the fourth PCR-amplified DNA fragment during the construction of the reference plasmid for detection of meat according to the present invention.

FIG. 5 is the sequencing confirmation of the reference plasmid for detection of meat according to the present invention.

FIG. 6 is the sequencing confirmation of the reference plasmid for detection of meat according to the present invention.

### DETAILED DESCRIPTION OF THE INVENTION

The present invention develops a reference plasmid for use in specifically detecting the meat of pig, cattle, sheep, deer, horse and kangaroo. The reference plasmid can be used in rapidly identifying the species of meat and can be used in testing commercial meat products. The present invention also sequences the gene sequences for detection and constructs the novel plasmid of the present invention by using these sequences.

The plasmid of the present invention can be used in detecting the meat of pig, cattle, sheep, deer, horse and kangaroo.

In accordance with the present invention, the meat internal control gene is a gene widely existing in animal meat, and is used for confirming that the tested sample is animal meat. Preferably, the meat internal control gene is the 18 S ribosomal RNA gene and the myostatin gene. More preferably, the meat internal control gene is the myostatin gene. Even more preferably, the meat internal control gene has a sequence as shown in SED ID NO:1.

In accordance with the present invention, the porcine growth hormone gene in the plasmid of the present invention is used as the identification gene for the meat of pig species. The identification gene for the meat of pig species known in the art includes the D-loop mtDNA, the mitochondrial cytochrome b gene, the 12S ribosomal RNA gene, the new DNA-specific porcine repetitive element, the porcine growth hormone gene, the short interspersed elements, the long interspersed repetitive elements and the satellite gene, etc. It has been surprisingly found in the present invention that the porcine growth hormone gene is suitable for constructing the plasmid of the present invention, with a better effect in identification of the meat of pig species. Preferably, the porcine growth hormone gene has a sequence as shown in SED ID NO:2.

In accordance with the present invention, the bovine 12S ribosomal RNA gene in the plasmid of the present invention is used as the identification gene for the meat of cattle species. The identification gene for the meat of cattle species known in the art includes the mitochondrial ATPase 8-ATPase 6 gene, the mitochondrial cytochrome b gene and the 12S ribosomal RNA gene, the short interspersed elements, the satellite DNA and the phosphodiesterase gene. It has been surprisingly found in the present invention that the bovine 12S ribosomal RNA gene is particularly suitable for constructing the plasmid of the present invention, with a better effect in identification of the meat of cattle species. Preferably, the bovine 12S ribosomal RNA gene has a sequence as shown in SED ID NO:3.

In accordance with the present invention, the ovine satellite DNA in the plasmid of the present invention is used as the identification gene for the meat of sheep species. The identification gene for the meat of sheep species known in the art includes the mitochondrial cytochrome b gene, the 12S ribosomal RNA gene and the satellite DNA. It has been surprisingly found in the present invention that the satellite DNA is particularly suitable for constructing the plasmid of the present invention, with a better effect in identification of the meat of sheep species. Preferably, the ovine satellite DNA has a sequence as shown in SED ID NO:4.

In accordance with the present invention, the cervine mitochondrial cytochrome b gene in the plasmid of the present invention is used as the identification gene for the meat of deer species. The identification gene for the meat of deer species known in the art includes the mitochondrial cytochrome b gene and the satellite DNA. It has been surprisingly found in the present invention that the cervine mitochondrial cytochrome b gene is particularly suitable for constructing the plasmid of the present invention, with a better effect in identification of the meat of deer species. Preferably, the cervine mitochondrial cytochrome b gene has a sequence as shown in SED ID NO:5.

In accordance with the present invention, the equine 12S ribosomal RNA gene in the plasmid of the present invention is used as the identification gene for the meat of horse species. The identification gene for the meat of horse species known in the art includes the mitochondrial cytochrome b gene and the satellite DNA. It has been surprisingly found in the present invention that the equine 12S ribosomal RNA gene can also be used as the identification gene for the meat of horse species, and is particularly suitable for constructing the plasmid of the present invention, with a better effect in identification of the meat of horse species. Preferably, the equine 12S ribosomal RNA gene has a sequence as shown in SED ID NO:6.

In accordance with the present invention, the kangaroo 12S ribosomal RNA gene in the plasmid of the present invention is used as the identification gene for the meat of kangaroo species. The identification gene for the meat of kangaroo species has not been disclosed in the art. It has been surprisingly found in the present invention that the kangaroo 12S ribosomal RNA gene can be used as the identification gene for the meat of kangaroo species, and is particularly suitable for constructing the plasmid of the present invention, with a better effect in identification of the meat of kangaroo species. Preferably, the kangaroo 12S ribosomal RNA gene has a sequence as shown in SED ID NO:7.

The present invention further provides a vector which comprises the plasmid of the present invention. In addition, the present invention further provides a host cell which comprises the vector of the present invention.

The present invention also relates to primers and probes for detecting the meat of pig, cattle, sheep, deer, horse and kangaroo. With the primers and probes of the present invention, the meat of pig, cattle, sheep, deer, horse and kangaroo can be detected.

The present invention provides a probe hybridized to the porcine growth hormone gene sequence, the sequence of which probe is CCTCAATACTCCAGAACCCCTCATTTTCCTC (SEQ ID NO:8). The probe can be used in detecting the meat of pig species.

The present invention provides a primer pair hybridized to the bovine 12S ribosomal RNA gene sequence, the sequences of which pair are ACATTCTCTACCCAAGAGAATCAAGC (SEQ ID NO:9) and TCCTCTCATGTAGCTAGTGCGTTTA (SEQ ID NO:10) respectively. The present invention further provides a probe hybridized to the bovine 12S ribosomal RNA gene sequence, the sequence of which probe is CCCTCCTCAAATAGATTCAGTGCATCTAACCCT (SEQ ID NO:11). The primers and probe can be used in detecting the meat of cattle species.

The present invention provides a primer pair hybridized to the ovine satellite DNA sequence, the sequences of which pair are CCTCTCCAGTGCTGACTTGGA (SEQ ID NO:12) and AAGCATGACATFGCTGCTAAGTTC (SEQ ID NO:13) respectively. The present invention further provides a probe hybridized to the ovine satellite DNA sequence, the sequence of which probe is CACGTGCATGCCCCCTCTCGA (SEQ ID NO:14). The primers and probe can be used in detecting the meat of sheep species.

The present invention provides a primer pair hybridized to the cervine mitochondrial cytochrome b gene sequence, the sequences of which pair are CATTTATTATCGCAGCACTCGCT (SEQ ID NO:15) and AGGTCTGGTACGAATAATACTAGTGAT (SEQ ID NO:16) respectively. The present invention further provides a probe hybridized to the cervine mitochondrial cytochrome b gene sequence, the sequence of which probe is CCACTTACTCTTCCTCCACGAAACAGGA (SEQ ID NO:17). The primers and probe can be used in detecting the meat of deer species.

The present invention provides a primer pair hybridized to the equine 12S ribosomal RNA gene sequence, the sequences of which pair are GATGGAGAGAAATGGGCTACATTTT (SEQ ID NO:18) and ACTGCTAAATCCTCCTTTAGTCTCCAG (SEQ ID NO:19) respectively. The present invention further provides a probe hybridized to the equine 12S ribosomal RNA gene sequence, the sequence of which probe is ACCCTAAGAACAAGAACTTTAACCCGGACGA (SEQ ID NO:20). The primers and probe can be used in detecting the meat of horse species.

The present invention provides a primer pair hybridized to the kangaroo 12S ribosomal RNA gene sequence, the sequences of which pair are GAGCTTAATTGAAACAGGCA (SEQ ID NO:21) and ACTTTTCTCCTCTTTTGTATTCC (SEQ ID NO:22) respectively. The present invention further provides a probe hybridized to the kangaroo 12S ribosomal RNA gene sequence, the sequence of which probe is TCCTCGACAAAACCTTAC (SEQ ID NO:23). The primers and probe can be used in detecting the meat of kangaroo species.

In accordance with the present invention, product-specific primers and probes suitable for detection are designed separately with respect to the specific regions of the abovementioned six gene sequences. Then the resulting DNA sequence fragments of the abovementioned primers are extended in both directions according to the gene DNA sequence data to design the respective primers, in order to obtain larger product-specific DNA fragments. Primer and probe techniques can be designed separately or with reference to the following documents: Chikuni, K., Tabata, T., Kosugiyama, M., Monma M. and Saito, M. 1994. Polymerase chain reaction assay for detection of sheep and goat meats. Meat Sci. 37:337-345; Meyer, R. Candrian, U. and Luthy, J. 1993. Detection of pork in heated meat products by the polymerase chain reaction. J. AOAC Int. 77:617-622; and Laube, I., Butschke, A., Zagon, J., Spiegelberg, A., Schauzu, M., Bögl, K. W., Kroh, L. W. and Broll, H. Detection method to identify beef in foods by the TaqMan™ technology. Bundesgesundheitsblatt Gesundheitsforsch. Gesundheitsschutz. pp.1-25. The species specificity is one consideration in designing the primers of the present invention, and the necessity to combine with the real-time PCR (TaqMan Fluorescence Probe System) is another important one. Therefore, the principle of designing the respective PCR primer resides in that its amplified product is less than 150 bp in order to have a positive effect on the graphical tendency of the subsequent probe fluorescence amplification plot. If it is more than 150 bp, the amplification plot of fluorescence may not be sharp enough, causing difficulty in determination or quantification.

Respective DNA fragments resulting from the PCR amplification are linked to each other, and then transferred into a proper vector, and after the steps of mass culture, extraction and purification, a large number of copied plasmids of the present invention can be obtained. The DNA transfer technique is well known by those skilled in the art, and any proper vector and commercial kit can be used in constructing the plasmid of the present invention. Preferably, pGEM®-T Easy Vector is used. Furthermore, any host cell suitable for the mass culture of the plasmid of the present invention can be used in the invention. These vectors include, but are not limited to, microorganisms and yeast. A preferable host cell is E. coli JM109.

The present invention further provides a kit comprising a linear plasmid DNA of the present invention, primers and probes which may be specifically hybridized to the porcine growth hormone gene, bovine 12S ribosomal RNA gene, ovine satellite DNA, cervine mitochondrial cytochrome b gene, equine 12S ribosomal RNA gene, kangaroo 12S ribosomal RNA gene and meat internal control gene, and a real-time PCR reaction solution formulation. The kit of the present invention further comprises suitable reaction buffers and DNA correction data. In accordance with the present invention, the PCR reaction enzyme and reagent used for the kit are well known by those skilled in the art. Other elements and preparations of the present kit can be made by those skilled in the art through modifying the general technique for preparing a PCR kit according to the conventional techniques.

The plasmid provided in the present invention is a standardized plasmid, which can be used in detecting the porcine growth hormone gene, the bovine 12S ribosomal RNA gene, the ovine satellite DNA, the cervine mitochondrial cytochrome b gene, the equine 12S ribosomal RNA gene and the kangaroo 12S ribosomal RNA gene. The plasmid constructed by the present invention can be prepared without any limitation, and can be simultaneously used as a reference substance for detection to solve the problem of lacking reference standards in identifying the species of meat products.

### EXAMPLE

### Example 1

### Materials and Method for Constructing the Plasmid of the Present Invention

1. Meat Source

Beef, beef jerky, pork, pork jerky, mutton and venison were all bought from the supermarkets of Taipei. The kangaroo jerky was bought from the supermarkets of Australia. Additionally, the horse blood and the sheep blood were directly bought from Taiwan.

2. DNA Extraction and Purification Kit

DNeasy® Tissue Kit (Qiagen, Hilden, Germany).

3. PCR Primer, Probe and Reaction Agent

The present invention designs species-specific primers and TaqMan probes for real-time PCR detection according to the DNA sequence comparison data, as shown in Table 1. In addition, the design of the primers for construction (tailed primers) is shown in Table 2 and the synthesis thereof is entrusted to TIB Molbiol (Berlin, Germany). The 5′-end of the probes employs the 6-carboxy-fluorescein label, and the 3′-end employs the 6-carboxytetramethyl-rhodamine label. The DNA Polymerase kit (PROtech Technologies, Inc., Taiwan) is a qualitative PCR reaction kit. The real-time PCR reaction kit is the LightCycler-FastStart DNA Master Hybridization Probes (Roche Applied Science, Mannheim, Germany).

TABLE 1

| | | | Amplicon |
|---|---|---|---|
| Primer/Probe | Sequence 5′-3′ | Specificity | (bp) |
| Deer | | | |
| Deer F | CATTTATTATCgCAgCACTCgCT | cyt(b)/sense | |
| Deer R | AggTCTggTACgAATAATACTAgTgAT | cyt(b)/antisense | 190 |
| Deer P | FAM- | | |
| | CCACTTACTCTTCCTCCACgAAAC | | |
| | AggA-TAMRA | | |
| Horse | | | |
| HosF2 | gATggAgAgAAATgggCTACATTTT | 12S rRNA/sense | |
| HosR2 | ACTgCTAAATCCTCCTTTAgTCTCCAg | 12S rRNA/antisense | 99 |
| HosP | FAM- | | |
| | ACCCTAAgAACAAgAACTTTAAC | | |
| | CCgg ACgA-TAMRA | | |
| Sheep | | | |
| SGF | CCTCTCCAgTgCTgACTTggA | satellite/sense | |
| SGR | AAgCATgACATTgCTgCTAAgTTC | satellite/antisense | 123 |
| SGP | FAM-CACgTgCATgCCCCCTCTCgA- | | |
| | TAMRA | | |
| Cattle | | | |
| BF | ACATTCTCTACCCAAgAgAATCAAgC | 12S/sense | |
| BR | TCCTCTCATgTAgCTAgTgCgTTTA | 12S/antisense | |
| BP | FAM- | | 193 |
| | CCCTCCTCAAATAgATTCAgTgCATCT | | |
| | AACCCT-TAMRA | | |
| Kangaroo | | | |
| KanF | gAgCTTAATTgAAACAggCA | 12S/sense | |
| KanR | ACTTTTCTCCTCTTTTgTATTCC | 12S/antisense | 106 |
| KanP | FAM-TCCTCgACAAAACCTTAC- | | |
| | TAMRA | | |
| Pig | | | |
| SWF | TCAgTTTACACTCACCTgATAgCATCT | growth hormone/sense | |
| SWR | gggTggTggAgAggggTgAATT | growth | 108 |
| SWP | FAM- | hormone/antisense | |
| | CCTCAATACTCCAgAACCCCTCA | | |
| | TTTT CCTC-TAMRA | | |
| Myostatin | | | |
| MYF | TTgTgCAAATCCTgAgACTCAT | myostatin/sense | |
| MYR | ATACCAgTgCCTgggTTCAT | myostatin/antisense | 97 |
| MYP | FAM- | | |
| | CCCATgAAAgACggTACAAggTATACT | | |
| | g-TAMRA | | |

TABLE 2

| Primer for | | |
|---|---|---|
| Construction | | |
| (Tailed | | |
| Primer) | Sequence 5′-3′ | Amplicon |
| Myostatin | | |
| MY-1 | TTgTgCAAATCCTgAgACTCAT | (A) |
| M-B | TgATgggTAggAgTATACCAgTgCCTg | |
| Cattle | | |
| B-M | CAggCACTggTATACATTCTCTACACCA | (B) |
| B-P | TCTCCACCACCCTCCTCTCATgTAgCT | |
| Pig | | |
| P-B | gAAATATTTAAAAACAgggTggTggAgA | (C) |
| P-K | gTTTCAATTAAgCTCTCAgTTTACACTCAC | |
| Kangaroo | | |
| K-P | gTgAgTgTAAACTgAgAgCTTAATTgAAAC | (D) |
| K-G | AgCACTggAgAggACTTTTCTCCTCTT | |
| Sheep | | |
| G-K | AAgAggAgAAAAgTCCTCTCCAgTgCT | (E) |
| G-H | CATTTCTCTCCATCAAgCATgACATTgC | |
| Horse | | |
| H-G | gCAATgTCATgCTTgATggAgAgAAATg | (F) |
| H-D | TgCgATAATAAATgACTgCTAAATCCTC | |
| Deer | | |
| D-H | gAggATTTAgCAgTCATTTATTATCgCA | (G) |
| Deer R6 | AggTCTggTACgAATAATACTAgTgAT | |

4. DNA Purification and Transfer Kit and Plasmid Extraction Kit

The recovery and purification of DNA amplification products of PCR employs the QIAquick Gel Extraction Kit (Qiagen, Hilden, Germany). The transfer system employs the Promega pGEM®-T Easy Vector (3015 bp) (Promega, Madison, Wis., USA). The plasmid DNA extraction employs the Qiagen Plasmid Mini Kit (Qiagen, Hilden, Germany).

5. PCR Reactions and Analysis of the Products

1. The First PCRs

PCR reaction solution:
10× PCR buffer . . . 5.0 μL
25 mM MgCl₂ . . . 4.0 μL
AmpliTaq DNA polymerase (5 U/μL) . . . 1 μL
2.5 mM dNTP . . . 4 μL
5 μM Primer F . . . 4 μL
5 μM Primer R . . . 4 μL
Template DNA (total 100 ng) . . . 5.0 μL
Aseptic pure water . . . 23 μL
Total volume . . . 50.0 μL

PCR conditions:

Myostatin Gene (Internal Control Gene)

| Step | Temperature | Time |
|---|---|---|
| 1. initial denaturation | 94° C. | 5 min |
| 2. denaturation | 94° C. | 30 sec |
| 3. annealing | 54° C. | 30 sec |
| 4. extension | 72° C. | 30 sec |
| Step 2 to Step 4, 35 circular reactions in total | | |
| 5. final extension | 72° C. | 7 min |
| cooling | 4° C. | |

Species-Specific PCR

| Step | Temperature | Time |
|---|---|---|
| 1. initial | 94° C. | 5 min |
| denaturation | | |
| 2. denaturation | 94° C. | 30 sec |
| 3. annealing | 60° C. | 30 sec |
| 4. extension | 72° C. | 30 sec |
| Step 2 to Step 4, 35 circular reactions in total | | |
| 5. final extension | 72° C. | 7 min |
| cooling | 4° C. | |

2. The Second PCRs

PCR reaction solution:
10× PCR buffer . . . 5.0 μL
25 mM MgCl₂ . . . 4.0 μL
AmpliTaq DNA polymerase (5 U/μL) . . . 1 μL
2.5 mM dNTP . . . 4 μL
10 μM Primer F . . . 2 μL
10 μM Primer R . . . 2 μL
Purified DNA (10× diluted) . . . 1 μL
Aseptic pure water . . . 31 μL
Total volume . . . 50.0 μL

PCR conditions:

| Step | Temperature | Time |
|---|---|---|
| 1. initial denaturation | 94° C. | 5 min |
| 2. denaturation | 94° C. | 30 sec |
| 3. annealing | 45° C. | 30 sec |
| 4. extension | 72° C. | 30 sec |
| Step 2 to Step 4, 5 circular reactions in total | | |
| 5. denaturation | 94° C. | 30 sec |
| 6. annealing | 60° C. | 30 sec |
| 7. extension | 72° C. | 30 sec |
| Step 5 to Step 7, 30 circular reactions in total | | |
| 8. final extension | 72° C. | 7 min |
| cooling | 4° C. | |

3. The Third PCR

PCR reaction solution:
10× PCR buffer . . . 5.0 μL
25 mM MgCl₂ . . . 4.0 μL
AmpliTaq DNA polymerase (5 U/μL) . . . 1 μL
2.5 mM dNTP . . . 4.0 μL
10 μM Primer F . . . 2.0 μL
10 μM Primer R . . . 2.0 μL
10 Purified DNA (10× diluted) . . . 1+1+1 μL
Aseptic pure water . . . 29 μL
Total volume . . . 50.0 μL

PCR conditions:

| Step | Temperature | Time |
|---|---|---|
| 1. initial | 94° C. | 5 min |
| denaturation | | |
| 2. denaturation | 94° C. | 30 sec |
| 3. annealing | 60° C. | 30 sec |
| 4. extension | 72° C. | 30 sec |
| Step 2 to Step 4, 35 circular reactions in total | | |
| 5. final extension | 72° C. | 7 min |
| cooling | 4° C. | |

4. The Fourth PCR

PCR reaction solution:

| | | |
|---|---|---|
| 10× PCR buffer | 5.0 | μL |
| 25 mM MgCl₂ | 4.0 | μL |
| AmpliTaq DNA polymerase (5 U/μL) | 1 | μL |
| 2.5 mM dNTP | 4.0 | μL |
| 10 μM Primer F | 2.0 | μL |
| 10 μM Primer R | 2.0 | μL |
| Purified DNA (10x diluted) | 1 + 1 + 1 | μL |
| Aseptic pure water | 29 | μL |
| Total volume | 50.0 | μL |

PCR conditions:

| Step | Temperature | Time |
|---|---|---|
| 1. initial denaturation | 94° C. | 5 min |
| 2. denaturation | 94° C. | 30 sec |
| 3. annealing | 60° C. | 30 sec |
| 4. extension | 72° C. | 30 sec |
| Step 2 to Step 4, 35 circular reactions in total | | |
| 5. final extension | 72° C. | 7 min |
| cooling | 4° C. | |

5. Real-Time PCR—Roche LightCycler

PCR reaction solution:

| | |
|---|---|
| Master mix | 2 μL |
| 25 mM MgCl₂ | 2.4 μL |
| 5 μM Primer F | 1.5 μL |
| 5 μM Primer R | 1.5.μL |
| 3.3 μM probe | 1.5 μL |
| Template DNA | 5.0 μL |
| Aseptic pure water | 6.1 μL |
| Total volume | 20.0 μL |

PCR conditions:

| Step | Temperature | Time |
|---|---|---|
| 1. initial denaturation | 95° C. | 10 min |
| 2. denaturation | 95° C. | 5 sec |
| 3. annealing | 60° C. | 25 sec |
| 4. extension | 72° C. | 8 sec |
| Step 2 to Step 4, 45 circular reactions in total | | |
| 5. cooling | 35° C. | 40 sec |

6. Recovery, Purification and Plasmid Extraction of PCR-Amplified DNAs

The PCR-amplified products are cut out of the gel (Agarose Gel) with a small knife, and then the DNAs are recovered and purified according to the kit operating steps. The extraction of the plasmid is also carried out with reference to the kit operating steps.

7. Plasmid Transfer

The plasmid transfer of the PCR-amplified products of the present invention is carried out with reference to the Promega pGEM®-T Easy Vector 3015 bp (Promega, Madison, Wis., USA) kit operating steps.

### Example 2

### Construction and Confirmation of the Plasmid of the Present Invention

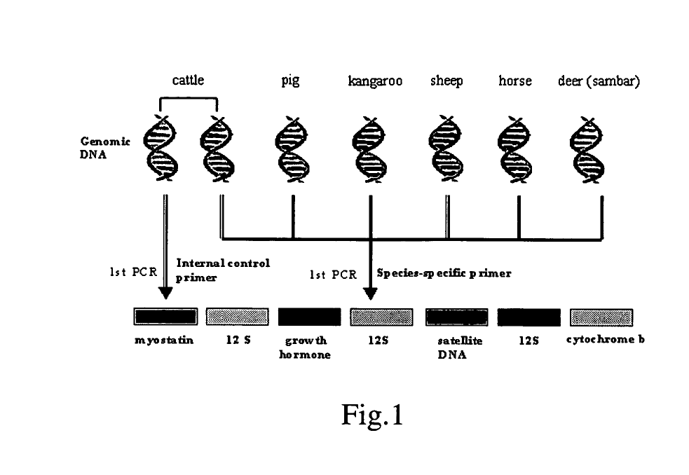

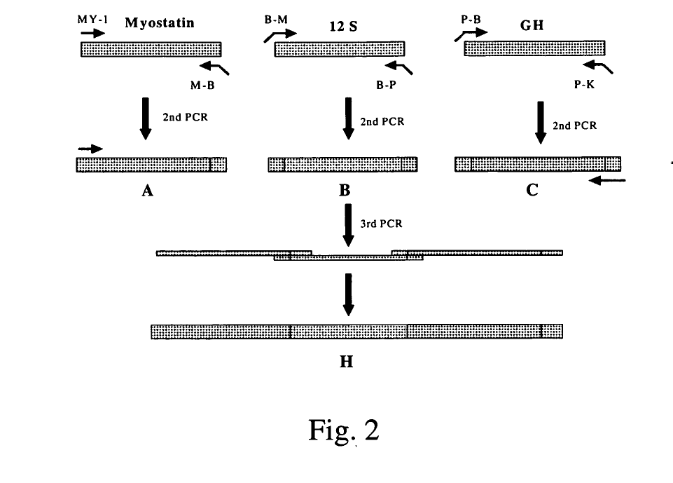

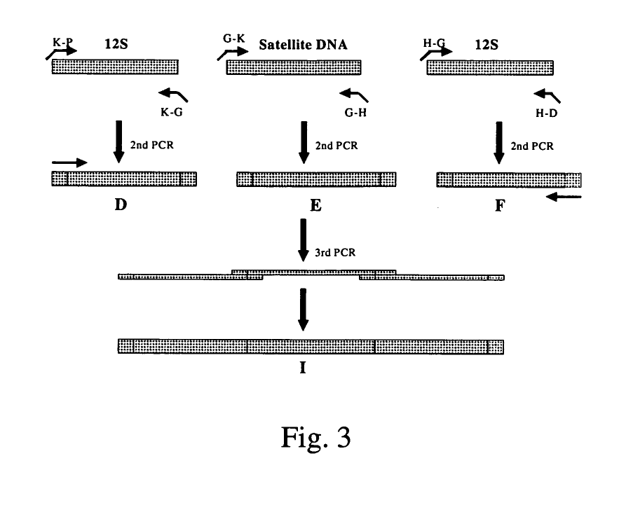

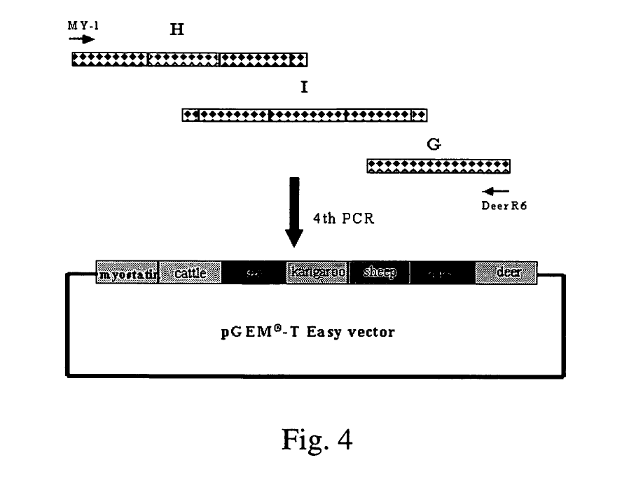

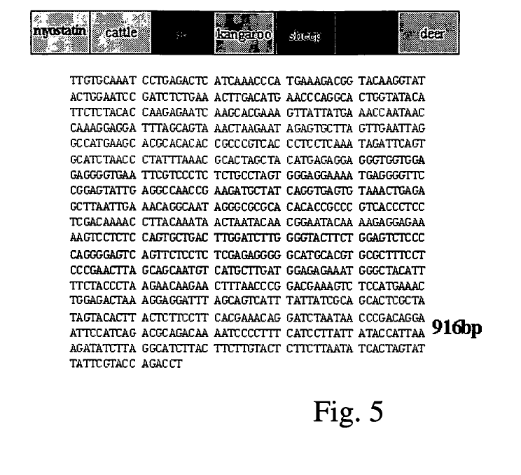

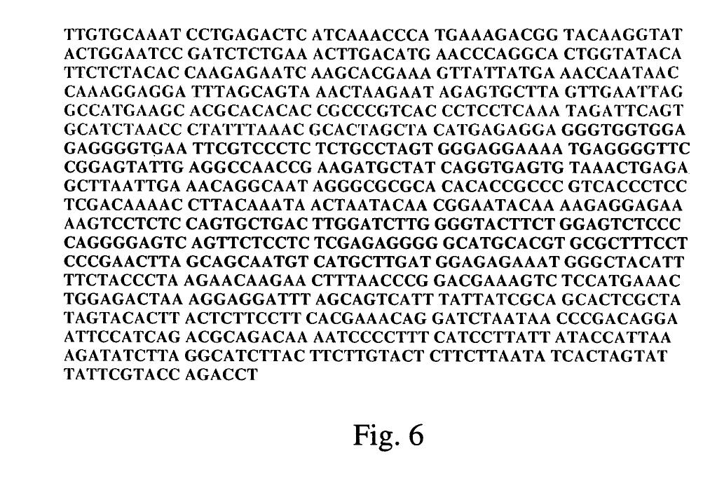

The specific DNA fragments of six meat types (cattle, pig, kangaroo, sheep, horse, and deer) and the DNA fragment of the myostatin gene, the meat internal control gene, are linked together by using the PCR method in the invention. The experiment steps are as follows (FIG. 1 to FIG. 4): first, with the genomic DNA of each meat type used as a template, and the genomic DNA of cattle taken as a template of the internal control gene, the first PCRs are carried out respectively by using the primer pairs (Table 1) (see Example 1 for the PCR reaction solution and PCR conditions), and seven amplified DNA fragments can be obtained respectively (FIG. 1); then, with the reaction products recovered as templates, the second PCRs are carried out by using the primer pairs (Table 2) (see Section 8 for the PCR reaction solution and PCR conditions), and seven fragments (A, B, C, D, E, F, G) can be obtained respectively; with the products recovered as templates, the third PCRs are carried out with the three fragments A, B, and C by using the primer pair MY-1 and P-K (Table 2 and FIG. 2) (see Section 8 for the PCR reaction solution and PCR conditions), and the fragment H can be obtained, and further, the third PCR is carried out with the three fragments D, E, and F by using the primer pair K-P and H-D (Table 2 and FIG. 3), and the fragment I can be obtained; then, the H and the I products are recovered. Finally, the fragment resulting from the fourth PCR carried out by using the primers MY-1 and Deer R6 with the fragments H, I, and G used as templates is the very DNA fragment to be constructed by the experiment, and then the fragment (916 bp) is transferred into the pGEM®-T Easy vector. The transferred fragment of gene also needs a sequencing analysis, in order to confirm that each of the seven PCR-amplified product fragments exists in the plasmid as a single copy (FIG. 5 and FIG. 6).

### Example 3

### Test of the Constructed Plasmid of the Present Invention

The DNA fragment to be constructed is subjected to the real-time PCR test before and after it is introduced into the plasmid, so as to ensure the reactions go well. In addition, more than five plasmids are selected for cloning after the DNA fragments are introduced therein, and well conserved. After the DNA sequencing confirmation, the plasmid is subjected to mass culture, extraction, purification, enzyme cut and recovery and purification, and then the DNA concentration is measured and a series of dilutions are carried out, and finally, the linear plasmid DNAs of a proper concentration are taken as the reference substance. In practice, the reference plasmid is subjected to the real-time PCR test to confirm it can react with all of the six species and the internal control gene.

In sum, the present experiment constructs the species-specific (cattle-, pig-, horse- sheep-, deer-, and kangaroo-specific, respectively) synchronous PCR-amplified product fragments (target genes) and the synchronous PCR-amplified product fragment of the myostatin gene (meat internal control gene), seven fragments in total, together on the plasmid as the reference substance, to solve the problems of the reference substance source and the preparation complexity. After the constructed plasmid is subjected to mass culture, copying and recovery and purification, the six species-specific (cattle-, pig-, kangaroo-, sheep-, horse- and deer-specific, respectively) DNA fragments and the myostatin internal control gene in the constructed plasmid are tested by the real-time PCR, to confirm there is no failure in the reactions.

What is claimed is:

1. A plasmid for identifying the species of meat, comprising a meat internal control gene and the following nucleic acid sequences: the porcine growth hormone gene, the bovine 12S ribosomal RNA gene, the ovine satellite DNA, the cervine mitochondrial cytochrome b gene, the equine 12S ribosomal RNA gene and the kangaroo 12S ribosomal RNA gene.

2. The plasmid as claimed in claim 1, wherein the meat internal control gene is the myostatin gene.

3. The plasmid as claimed in claim 2, wherein the myostatin gene has a sequence as shown in SEQ ID NO:1.

4. The plasmid as claimed in claim 1, wherein the porcine growth hormone gene has a sequence as shown in SEQ ID NO:2.

5. The plasmid as claimed in claim 1, wherein the bovine 12S ribosomal RNA gene has a sequence as shown in SEQ ID NO:3.

6. The plasmid as claimed in claim 1, wherein the ovine satellite DNA has a sequence as shown in SEQ ID NO: 4.

7. The plasmid as claimed in claim 1, wherein the cervine mitochondrial cytochrome b gene has a sequence as shown in SEQ ID NO:5.

8. The plasmid as claimed in claim 1, wherein the equine 12S ribosomal RNA gene has a sequence as shown in SEQ ID NO:6.

9. The plasmid as claimed in claim 1, wherein the kangaroo 12S ribosomal RNA gene has a sequence as shown in SEQ ID NO:7.

10. A vector, comprising the plasmid as claimed in claim 1.

11. A host cell, comprising the vector as claimed in claim 10.

12. The plasmid as claimed in claim 1, which can be used in detecting the meat of pig, cattle, sheep, deer, horse and kangaroo.

13. A probe hybridized to the porcine growth hormone gene sequence, having a sequence as shown in SEQ ID NO: 8.

14. A primer pair hybridized to the bovine 12S ribosomal RNA gene sequence, having sequences as shown in SEQ ID NO: 9 and SEQ ID NO:10.

15. A probe hybridized to the bovine 12S ribosomal RNA gene sequence, having a sequence as shown in SEQ ID NO:11.

16. A primer pair hybridized to the ovine satellite DNA sequence, having sequences as shown in SEQ ID NO: 12 and SEQ ID NO:13.

17. A probe hybridized to the ovine satellite DNA sequence, having a sequence as shown in SEQ ID NO:14.

18. A primer pair hybridized to the cervine mitochondrial cytochrome b gene sequence, having sequences as shown in SEQ ID NO:15 and SEQ ID NO:16.

19. A probe hybridized to the cervine mitochondrial cytochrome b gene sequence, having a sequence as shown in SEQ ID NO:17.

20. A primer pair hybridized to the equine 12S ribosomal RNA gene sequence, having sequences as shown in SEQ ID NO:18 and SEQ ID NO:19.

21. A probe hybridized to the equine 12S ribosomal RNA gene sequence, having a sequence as shown in SEQ ID NO:20.

22. A primer pair hybridized to the kangaroo 12S ribosomal RNA gene sequence, having sequences as shown in SEQ ID NO:21 and SEQ ID NO:22.

23. A probe hybridized to the kangaroo 12S ribosomal RNA gene sequence, having a sequence as shown in SEQ ID NO:23.

24. A method for identifying the species of meat, which comprises using the plasmid as claimed in claim 1 and one or more primers and probes as claimed in claim 13.

25. A kit for identifying the species of meat, comprising the plasmid as claimed in claim 1, one or more primers and probes specifically hybridized to the meat internal control gene, the porcine growth hormone gene, the bovine 12S ribosomal RNA gene, the ovine satellite DNA, the cervine mitochondrial cytochrome b gene, the equine 12S ribosomal RNA gene and the kangaroo 12S ribosomal RNA gene, and a real-time PCR reaction solution formulation.
